(19) United States
(12) Patent Application Publication
(10) Pub. No.: US 2004/0105895 A1
(43) Pub. Date: Jun. 3, 2004

(54) Monovalent-selective cation exchangers as oral sorbent therapy

(76) Inventor: Stephen R Ash, Lafayette, IN (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/467,229

(22) PCT Filed: Feb. 6, 2002
(86) PCT No.: PCT/US02/03474

(30) Foreign Application Priority Data
Feb. 6, 2001 (US) 60266759

(51) Int. Cl. A61K 33/24

(52) U.S. Cl. 424/617

(57) ABSTRACT

The present invention relates to compounds and methods of treating patients exhibiting high levels serum toxins. The present invention finds particularly advantageous use for patients suffering from renal and/or liver dysfunction. The present invention includes administering to such patients a zirconium-silicate sorbent in amounts sufficient to reduce one or more of the levels of the serum toxins. The zirconium-silicate sorbent can function as a cation exchanger and exchange one or more cations and adsorb cationic toxics, such as, ammonium cations, potassium cations, sodium cations, calcium cations, magnesium cations, from the patient. Additionally, the zirconium-silicate sorbent can be combined with one or more of a carbon agent (or charcoal), zinc oxide and/or agent to enhance the intestine permeability such as a non absorbable alcohol.

### CROSS-REFERENCE TO RELATED APPLICATION

The present application claims the benefit of U.S. Provisional Application Serial No. 60/266,759, filed Feb. 6, 2001, which is hereby incorporated by reference in its entirety.

### BACKGROUND OF THE INVENTION

In therapy for kidney and/or liver dysfunction it is necessary to facilitate removal of toxins from the body. The toxins can be cationic salts such as potassium, sodium and ammonium salts, or the toxins can be organic toxins such as urea, creatinine, uric acid, hippurates and homocysteine to name just a few. Dialysis is used to remove many of these toxins. Dialysis treatments severely limit a patent's core life functions. Furthermore, dialysis alone may not lower the concentration of serum toxins to acceptable levels.

The normal plasma potassium level is typically maintained at about 3.5 to 5 mEq/L, and toxicity can begin at levels of over 6 mEq/L. Excess potassium (hyperkalemia) can occur in patients with chronic renal dysfunction or failure before dialysis is implemented. It can also occur in patients on treatment regiments including three-times-per-week dialysis, especially on weekends when the interval between dialysis treatment is longer.

Current therapy for patients with hyperkalemia includes either dialysis and/or oral intake of 15 to 30 grams KAYEXELATE®, which contains polystyrene sulfonate sodium (PSS), several times per day until the potassium level decreases or dialysis can be implemented to remove potassium. The PSS is used to adsorb potassium from the intestine and colon. While the kidneys are the main excretory routes for potassium, it is well known that the concentration of potassium is much higher in the colon than in the small intestine or in the blood. Furthermore, the colon mucosa has an active transport pump for potassium that transports potassium from the peritoneal into the colon. This is similar to the potassium pump that exists in the distal tubule of the kidney. Unfortunately, it has been observed that PSS also exhibits a greater affinity for divalent cations, such as calcium and magnesium than for mono-valent cations. Consequently much of the capacity of the PSS for adsorbing cations is already exhausted by the time the PSS reaches the colon. This obviously reduces the effectiveness of administering PSS to treat hyperkalemia.

Additionally, PSS can aggregate into a solid mass and cause an obstruction within the intestines, intestinal ischemia and ulcers. These risks are increased in patients with diminished gut activity or constipation, which is symptomatic of patents with kidney dysfunction. To offset the risks of such obstruction and to increase potassium removal by the gut, PSS treatment is given in combination with a non-absorbable sugar, such as SORBITOL in an amount that assures the development of diarrhea in the patient. It is not surprising that many patients complain of the volume of SORBITOL needed, its sweet taste, and obviously, the diarrhea and accompanying abdominal discomfort.

Typically the blood urea level is also high with kidney dysfunction. The kidney is a principle organ for urea excretion and the liver is the only source of urea production. The balance of urea formation by the liver and kidney excretion normally maintains a blood urea nitrogen (BUN) level of about 13 to 20 mg per deciliter (mg %). Because of kidney dysfunction, the BUN level can range from about 50 to about 200 mg % depending upon the daily protein intake and/or degree of protein breakdown in the body. Urea is produced in the liver by transfer of nitrogen from amino acids, ammonium and other nitrogenous chemicals, and the urine excretion of urea is the major method of the body to remove excess nitrogen. Frequently, patients with decreased kidney capacity are forced to eat a diet with strict limitations in protein in an effort to lower their BUN level. Approximately 25% of the urea produced every day passes into the gut, where bacteria with the urease enzyme break it down into ammonium and carbonate. Most of the ammonium is absorbed by the gut and converted in the liver back into urea. Excess urea and ammonium in the gut can cause uremia (wide-ranging symptoms of kidney failure). However, removal of a significant amount of ammonium from the gut would significantly lower BUN levels and reduce the onset of uremia.

In addition, patients with liver dysfunction and/or failure also exhibit an elevation of blood level ammonium. Elevated ammonium has a rough correlation to brain dysfunction and liver failure encephalopathy (brain dysfunction). The standard therapy for hyperammonemia is the use of non-absorbable saccharide such as lactulose. However, this therapy is not very effective for decreasing blood level ammonium.

Currently extracorporeal dialysis is the preferred regimen to treat hyperammonemia and uremia Dialysis treatments are expensive, time-consuming, somewhat risky and make a normal lifestyle almost impossible. Furthermore dialysis treatment is not completely effective in lowering serum toxin concentrations to acceptable levels, particularly for patients with end stage renal disease. If toxin concentrations are normal after one dialysis treatment, they will be high before the next dialysis treatment.

In light of the above-described problems there is a continuing need for advancements in the relevant field, including improved methods for treating liver and kidney dysfunction and particularly for lowering serum toxin levels. The present invention is such an advancement and provides a wide variety of benefits and advantages.

### SUMMARY OF THE INVENTION

The present invention relates to pharmaceutical compositions and methods of treating patients having elevated levels of one or more serum toxins including, but not restricted to, patents with liver and renal dysfunction. Various aspects of the invention are novel, non-obvious and provide various advantages. While the actual nature of the invention covered herein can only be determined with reference to the claims appended hereto, certain forms and features, which are characteristic of the preferred embodiment disclosed herein, are described briefly as follows.

In embodiment the present invention provides a method of treating animals. The method involves selecting an animal capable of deriving a benefit from lower levels of one or more serum toxins. The selected animal is then treated by administering a pharmaceutical preparation that comprises a zirconium-silicate sorbent having exchangeable cations. The exchangeable cations can be selected to include hydronium cations, calcium cations, sodium cations, potassium cations, magnesium cations and mixtures thereof. Additionally, the animal can be treated with activated charcoal and/or zinc oxide to remove additional toxins from either serum and/or the gut. Additionally, the pharmaceutical preparation can include a non-absorbable alcohol to improve intestinal permeability. The pharmaceutical preparation can also include one or more of: diluents, carriers, favoring agents, wetting agents, lubricants, binders, and the like. The pharmaceutical preparation is administered to the animal in a unit dosage form that provides a therapeutically effective amount of the sorbent and optionally the therapeutic additive such as the charcoal, zinc oxide and/or a non-absorbable alcohol to reduce the level of one or more serum toxins. The pharmaceutical preparation can be formulated to be administered orally, rectally or through an ostomy inlet. The pharmaceutical preparation can be provided as a solid, i.e. a powder, a pill or a pellet; a liquid, i.e., a suspension, a gel, a paste, or a thick liquid. The pharmaceutical preparation can be administered alone as a pill, or as a suppository, or combined with food or drink.

In other embodiments, the present invention provides a method of treating a patient with abnormally high levels of one or more toxins. The method comprises administering to a patient a pharmaceutical preparation comprising a zirconium-silicate sorbent, and either in combination or separately an intestinal permeability enhancing agent, zinc oxide and/or activated charcoal. The zirconium-silicate sorbent has one or more adsorbed, exchangeable cations selected from hydronium cations, calcium cations, sodium cations, potassium cations, magnesium cations and mixtures thereof. Preferably, the zirconium-silicate sorbent is selected to selectively release or desorb cations such as calcium and hydronium into the patient and adsorb ammonium and potassium cations from the patient. Examples of intestinal permeability enhancing agents at low concentrations include alcohol, polyethylene glycol, glycerin, propylene glycol, acetone, and polyvinyl alcohol. The pharmaceutical composition can be administered either orally, through an ostomy inlet, or rectally.

In still other embodiments, the present invention provides a pharmaceutical composition in unit dosage form for treating patents having elevated levels of one or more toxins. The sorbent is administered to the patient in a therapeutically effective amount to reduce the serum concentration of one or more serum toxins. The composition comprises a monovalent cation exchanger that comprises a zirconium-silicate sorbent. The zirconium-silicate sorbent has exchangeable cations absorbed thereon. The exchangeable cations can be selected to include hydronium, calcium, sodium, potassium, magnesium and mixtures thereof. Additionally, the pharmaceutical composition can include a therapeutic agent including an activated charcoal or an intestinal tissue permeability-enhancing agent and optionally a carrier or diluent. The therapeutically effective amount of the pharmaceutical composition can be selected to be between about 0.015 g and about 1.5 g per kilogram of body weight. In preferred embodiments, the monovalent cation exchanger comprises a mixture of calcium and hydronium cations absorbed thereon.

It is an object of the present invention to provide a pharmaceutical composition including a monovalent-selective cation exchanger as a sorbent for removal of serum toxins.

Further objects, features, aspects, forums, advantages and benefits will become apparent from the description and the drawings contained herein.

### BRIEF DESCRIPTION OF THE DRAWINGS

FIG. 1 is a graph illustrating the ammonium-binding ability of one embodiment of a zirconium-silicate sorbent (ZS) in accordance with the present invention, expressed in amount of ammonium bound in a physiologic solution containing calcium, magnesium, potassium and sodium, versus the concentration of ammonium in this solution. For comparison, ammonium binding in the same solution by a more standard cation exchanger, i.e., zirconium phosphate (ZP), are shown in the lower right of the graph.

FIG. 2 is a bar graph illustrating the average food consumption of rats alternatively fed a diet including a zirconium-silicate sorbent and a diet without any zirconium-silicate sorbent according to the experimental procedure discussed in Example 2.

FIG. 3 is a bar graph illustrating the average weight gain of the rats treated according to the experimental procedure discussed in Example 2.

FIG. 4 is a bar graph illustrating the daily excretion of urea nitrogen from rats treated according to the experimental procedure discussed in Example 2.

FIG. 5 is a bar graph illustrating the daily urinary potassium excretion from the rats treated in accordance with the experimental procedure described in Example 2.

FIG. 6 is a bar graph illustrating the average magnesium excreted from the rats treated in accordance with the experimental procedure discussed in Example 2.

FIG. 7 is a bar graph illustrating the daily excretion of ionized calcium from the rats treated in accordance with the experimental procedure discussed in Example 2.

FIG. 8 is a bar graph illustrating the average daily urinary sodium excretion from the rats treated in accordance with the experimental procedure described in Example 2.

FIG. 9 is a bar graph illustrating the average pH of urine excreted from rats treated in accordance with the experimental procedure described in Example 2.

### DETAILED DESCRIPTION OF THE INVENTION

For the purposes of promoting an understanding of the principles of the invention, reference will now be made to the embodiments illustrated herein and specific language will be used to describe the same. It will nevertheless be understood that no limitation of the scope of the invention is hereby intended. Any alterations and further modifications in the described processes, systems or devices, and any further applications of the principles of the invention as described herein, are contemplated as would normally occur to one skilled in the art to which the invention relates.

In general, the present invention relates to a method of treating patients exhibiting elevated levels of serum toxins. The elevated levels of serum toxins can be, but are not required to be, a result of liver or kidney dysfunction (or reduced function), drug overdose, drug interaction and/or trauma. The present invention provides particular advantages for patients suffering in the end stages of chronic renal and liver diseases. The elevated levels of serum toxins include, but are not limited to, ionic toxins such as ammonium, calcium, potassium, sodium, phosphate, as well as organic toxins including, for example, creatinine, urea, bile acids, bilirubin, aromatic amino acids, mercaptans and the like.

By use of the term patient, it is intended to include human as well as other animals particularly, but not restricted to domesticated mammals, such as, dogs, cats, and horses.

Preferably, the treatment includes administering a zirconium-silicate sorbent either alone or in combination with other therapeutically effective agents. Additionally, the zirconium-silicate sorbent can be combined with one or more pharmaceutically acceptable carriers, diluents, dispersing agents, lubricants, binders and the like. The method of administration can vary depending upon the patient history, disease etiology and patient and/or disease prognosis. The therapeutically effective agents can include activated carbon compounds, zinc oxide, and/or intestinal tissue permeability-enhancing agents.

The zirconium-silicate sorbent can be selected to include a wide variety of synthetic and natural cation exchangers. In the preferred embodiments, the cation exchangers can have a wide variety of porous sizes, shapes and ionic charges to allow them to effectively bind monovalent cations, minimizing the competitive binding or adsorption by di- and tri-valent cations. Specific examples of the preferred cation exchangers can be found and described in U.S. Pat. No. 5,338,527 entitled “Zirconium-Silicate Composition and Method of Preparation and Uses Thereof”; U.S. Pat. No. 5,888,472 entitled “Zirconium-Silicate Molecular Sieves and Process Using the Same”; U.S. Pat. No. 5,891,471 entitled “Zirconium-Silicate and Zirconium-Germanate Molecular Sieves and Process Using the Same”; U.S. Pat. No. 6,099,737 entitled “Process for Removing Toxins from Blood Using Zirconium Metallate or a Titanium Metallate Compositions”; and U.S. Pat. No. 6,332,985 entitled “Process for Removing Toxins from Bodily Fluids Using Zirconium or Titanium Microporous Compositions”, all which are incorporated by reference in their entirety.

The zirconium-silicate sorbent for use in the present invention has a microporous framework structure containing at least ZrO₃ octahedral units and SiO₂ tetrahedral units and an empirical formula on an anhydrous and as synthesized basis illustrated below in Equation 1:

AₚMₓZiₐ₋ₓSiₙOₘ (1)

where A is an exchangeable cation selected from the group consisting of calcium, magnesium, potassium, sodium, ammonium, hydronium or mixtures thereof, M is at least one framework metal selected from the group consisting of hafnium (Hf⁴⁺), tin (Sn⁴⁺), niobium (Nb⁵⁺), titanium (Ti⁴⁺), cerium (Ce⁴⁺), praseodymium (Pr⁴⁺), and terbium (Tb⁴⁺), “p” has a value from about 1 to about 6, “x” has a value from greater than zero to less than 1, “n” has a value from about 2 to about 4, “m” has a value from about 7 to about 12. In preferred embodiments, the sorbent is characterized in that it has an average pore diameter of less than about 8 Å. Optionally the zirconium-silicate sorbent can include within its framework GeO₂.

The zirconium-silicate sorbent of this invention can contain some of the alkali metal templating agent in the pores. These metals are described as exchangeable cations meaning that they can be exchanged for other (secondary) cations. Generally, the A exchangeable cations can be exchanged for other alkali metal cations (K⁺, Na⁺, Rb⁺, Cs⁺), alkaline earth cations (Mg²⁺, Ca²⁺, Sr²⁺, Ba²⁺), hydronium cation (H₃O⁺), ammonium cations (NH₄⁺) or mixtures thereof. The methods used to exchange one cation for another are well known in the art and involve contacting the sorbent with a solution containing the desired cation at exchange conditions. Typically the solid zirconium-silicate sorbent is suspended in an aqueous solution that also includes an excess of the cations to be exchanged for the cations that are already contained in the sorbent. Typically the desired cations are provided in about 2 to about 10 molar excess based upon the number of moles of cations (or available valences on the sorbent) already absorbed on the sorbent. The aqueous solution can be maintained at a temperature of between about 25° C. to about 100° C. and for a time of about 10 minutes to about 2 hours to allow the exchange reaction to go to completion. Thereafter the suspension is filtered to collect and retain the solid zirconium-silicate sorbent, which is then dried either at room temperature or under elevated temperatures, preferably not greater than about 150° C.

The zirconium-silicate sorbents of this invention have a framework structure of octahedral ZrO₃ units, at least one of tetrahedral SiO₂ units and tetrahedral GeO₂ units and optionally octahedral MO₃ units. This framework results in a microporous structure having an intracrystalline pore system with uniform pore diameters, i.e., the pore sizes are crystallographically regular. The diameter of the pores can vary considerably from about 3 Å and larger. The sorbents of this invention are also capable of selective ion exchange of ammonium cations and/or potassium cations.

In a preferred embodiment, the present invention provides a method of treating a patient in need of treatment by administering to the patient a pharmaceutical preparation comprising a zirconium-silicate sorbent. The zirconium-silicate sorbent is administered in a therapeutic amount effective to lower the concentration of one or more toxins.

One specific composition for use in the present invention includes a zirconium-silicate that includes a combination of calcium and hydronium ions absorbed thereon. The calcium and hydronium ions can be absorbed on the zirconium-silicate sorbent in a ratio (Ca²⁺:H₃O⁺) of between about 0.1 and about 0.9, more preferably in a ratio between about 0.4 and about 0.6. In specific embodiments, the zirconium-silicate sorbent is provided to have a cation capacity from between about 0.3 mEq. per gram of sorbent to about 0.6 mEq. per gram of sorbent.

The zirconium-silicate sorbent can be administered to the patient in a unit dosage through a variety of routes, including orally, rectally, and through an ostomy inlet. The zirconium-silicate sorbent can be provided in a form depending upon the patient's disease stage, mode of administration and the desired beneficial effect to the patient. For example, the zirconium-silicate sorbent can be provided as a pill, a powder, a viscous liquid, a gel or a suspension. The zirconium-silicate sorbent can be combined with one or more binders, diluents, flavoring agents, wetting agents, lubricants, and the like. The zirconium-silicate sorbent can be administered individually as a single medication such as a pill or a suppository as discussed below more fully. Additionally the zirconium-silicate can be admixed with food and/or drinks, or even baked within certain foods such as breads and cookies to facilitate administration.

The zirconium-silicate sorbent, in accordance with the present invention, can be administered to the patient in a therapeutically amount effective to reduce the level of one or more serum toxins. In preferred examples, the zirconium-silicate sorbent can be administered to the patient in a single unit dosage amount of between 1 gram and about 20 grams. The patient can be given multiple doses per day. Preferably a therapeutically effective amount of the sorbent can be selected to be between about 0.15 g and about 1.5 g per kilogram of body weight daily.

The zirconium-silicate of the present invention can provided as a solid in a wide range of particle sizes depending upon the desired mode of administration, co-additives, and considering the admixture of one or more diluents, carriers, lubricants and the like. In preferred embodiments, the zirconium-silicate of the present invention is provided as fine powder having an average particle size between about 3 to about 50 microns, more preferably between about 5 and about 10 microns.

In addition to reducing the concentration of one or more toxins, the pharmaceutical preparation of the present invention can increase the serum concentration of selected ionic components. Typically the electrolyte balance for patients must be maintained within certain narrow ranges. Deviation either by retaining too much of a particular component or by having too little of that same component can be equally detrimental and even life threatening to the patient. Maintaining a desired electrolyte balance for patients experiencing either renal or liver dysfunction can be particularly difficult.

In the preferred embodiment, the zirconium-silicate sorbent functions as a cation exchange. As such, it is preferable to pretreat or prepare the zirconium-silicate sorbent as above described to include cations which can be exchanged and potentially provide a benefit to the patient, as well as, removing undesirable cations to lower serum toxins from the patient. The pharmaceutical preparation provided according to the present invention can provide at least about 5 mg of calcium per kg of body weight, more preferably at least 30 mg of calcium per kg of body weight per dose. The patient can receive as many doses as medically expedient to increase concentration of the selected cation to within acceptable levels. In preferred embodiments, the zirconium-silicate sorbent of the present invention can increase the concentration of selected cations for example calcium or magnesium while at the same time reducing the concentration of ionic toxins, i.e., sodium, ammonium and/or potassium.

The zirconium-silicate sorbent can be administered in combination with one or more therapeutically effective additives, which can include a charcoal or carbon agent, zinc oxide, and/or an intestinal tissue permeability-enhancing agent. The zirconium-silicate sorbent and the one or more additives can be combined in a single pharmaceutical formulation. Alternatively, the zirconium-silicate sorbent and one or more of the additional agents can be provided in separate pharmaceutical formulations, which can be administered separately to the patient either through the same administration route or through a different administration route.

The carbon agent can be selected from a wide variety of commercially available pharmaceutically acceptable carbon sources. For example an activated carbon agent can be obtained in USP grade from Mallinckrodt, Inc. of St. Louis, Mo. and can be used in the present invention. The carbon agent can be administered to the patient in a therapeutic amount effect to lower the concentration of one or more toxins. The use of charcoal sorbents is discussed in Sinclair, A.; Babbs. C. F.; Griffin D. D.; and Ash S. R., “Roux-Y Intestinal Bypass for Administration of Sorbents In Uremia”, Kidney Int. Supple., 13(88): S153-S159, 1979, and in Sinclair, A,; Griffin, D. D. Voreis, J. D. and Ash, S. R. “Sorbent Binding of Urea and Creatinine in a Roux-Y Intestinal Segment,” Clin. Nephrol. 11(2): 97-104, 1979, each of which is incorporated by reference in its entirety.

In preferred embodiments, a single unit dose of an activated charcoal agent that can provide a therapeutic effect to the patient can be selected to be at least about 0.1 g of carbon per kg body weight per day, more preferably, at least about 0.2 g of carbon per kg body weight per day, and still yet more preferably, at least about 0.3 g of carbon per kg body weight per day. The amount of carbon should not exceed the amount that will promote constipation and/or create a blockage in the patient's intestine. Consequently, it is preferred to include less than about 3 g of carbon per kg body weight, more preferably, less than about 1.5 g of carbon per kg body weight per day.

The carbon can be administered to the patient through a variety of routes. For example, the carbon can be administered orally, rectally, or through an ostomy inlet. Obviously, the particular pharmaceutical formulation of the carbon can vary depending upon the mode of administration, the patient's history and the disease etiology. The carbon can be capsulated within a coating such as a cellulose or polymeric coating discussed below in more detail. Alternatively, the carbon can be entrained within a carrier or diluent to provide a liquid and/or gel suspension that can be administered to the patient. Additionally, the carbon can be combined in a single pharmaceutical preparation or a unit dosage form with the zirconium-silicate sorbent.

The carbon can bind to and/or otherwise adsorb a wide variety of toxins particularly organic toxins, which are found in detrimentally high serum concentrations often correlated with liver dysfunction or disease. Examples of toxins adsorbable on the carbon include, but are not restricted to, creatinine, bile acids, bilirubin, aromatic amino acids, mercaptans, phenols and homocysteine, uric acid and hippurates.

The zirconium-silicate sorbent of the present invention can also be combined with zinc oxide. Zinc oxide binds to phosphate (PO43+) salts found in the gastrointestinal track. For pharmaceutical uses acceptable sources of zinc oxide are preferably USP grade. For example, zinc oxide is commercially available from Southern Ionics, Inc. located in West Point MS. Excess phosphate (hyperphosphatemia, i.e. a serum phosphorus concentration >5 mg/L inorganic phosphorus level) is associated with renal failure. Consequently, many patients that are suffering from high levels of magnesium and potassium also exhibit high serum levels of phosphate.

A composition comprising zinc oxide can be administered with the pharmaceutical preparation containing zirconium-silicate sorbent according to the present invention. The zinc oxide composition can be combined with the zirconium-silicate sorbent in the pharmaceutical preparation of the present invention, which is then administered to the patient. Alternatively, the zinc oxide composition can be administered to the patient separately from the zirconium-silicate sorbent either through the same or a different administration route.

Zinc oxide can be administered to a patient in a therapeutic amount sufficient to lower the level of phosphate concentration in serum. Preferably, zinc oxide can be administered to a patients in an amount at least about 0.05 g per kilogram of body weight per day, more preferably at least about 0.1 g per kilogram of body weight per day, and still more preferably at least about 0.2 g per kilogram of body weight per day. As with all drug therapies too much of the drug can be detrimental to the patient. The amount of zinc oxide administered to the patient is less than the amount that will induce hypophosphatemia or other health risks. Typically, no more than about 5 g of zinc oxide per body weight per day is administered to the patient, more preferably less than about 3 g of zinc oxide is provided to the patient per kilogram body weight per day.

The zirconium-silicate sorbent of the present invention can be combined with an intestinal tissue permeability-enhancing agent. The intestinal tissue permeability-enhancing agent can be combined with carbon and/or zinc oxide or replace either or both of these agents in a pharmaceutical formulation for use in the present invention. In the preferred embodiments, the intestinal tissue permeability-enhancing agent is selected to include a non-absorbing agent such as ethanol, polyethylene glycol, glycerin, propylene glycol, acetone, and polyvinyl alcohol and mixtures of these agents. The intestinal tissue permeability-enhancing is selected to be substantially non absorbable by the intestine and is preferably administered to minimize absorption by the stomach. The intestinal tissue permeability-enhancing agent can be administered to the patient, similarly to the carbon agent, through a variety of administration routes, including orally, rectally, and through ostomy inlet. Examples of intestinal tissue permeability enhancing agents are described in Koszuta, J.; Carter, J. M.; and S. R. Ash “Effect of Ethanol Perfusion on Creatinine Removal in a Roux-Y Intestinal Segment,” Int'l. J. Artif Organs 14(7): 417-423, 1979, which is incorporated by reference in its entirety.

The pharmaceutical preparation containing zirconium-silicate sorbent according to the present invention can be combined with one or more of charcoal, zinc oxide and a intestinal tissue permeability-enhancing agent. Certain formulations provide particularly advantageous results for treating patients suffering from renal and/or liver dysfunction. Pharmaceutical formulations can be tailored to the patient's disease state, diet, activity level, and to enhance other existing or recommended treatment regiments, particularly, dialysis treatments. Administering one or more pharmaceutical formulation prepared according to the present invention serve to reduce the frequency of dialysis treatments. Additionally and possible more important from a patient's standpoint, present invention can allow a patient to ingest a more “normal diet”-other than taking one or more of the pharmaceutical preparations—and still significantly reduce the patient's toxin levels. The pharmaceutical preparation can be specifically formulated to correspond the patient' diet. This can facilitate better patient compliance with required treatment/medications and contribute to the patient's overall mental state and physical health.

It has unexpected been determined that a patient can tolerate a large volume or amount of the zirconium-silicate sorbent (and any admixed therapeutic additives). It has been determined as evidenced by the experiments described below that large amounts of the sorbent (and any admixed therapeutic additives) can be ingested without adverse effects. Furthermore, patients can ingest an amount of the sorbent (and therappeutic additive) equal to at least 10% by weight of their daily dietary intake still gain weight and not lose their appetite. Treating a patient with the pharmaceutical preparations of the present invention may not provide the same or equivalent clearance of toxins as a normal, healthy, functional kidney and/or liver. However, increasing the amount and efficiency of the clearance drugs, i.e. the sorbent and admixed additive of present invention, will increase the amount of toxins eliminated from the body. This will insure better patient compliance and reduce the reliance of dialysis to remove many toxins, which in turn significantly impacts a patient's life.

It has also been determined that the pharmaceutical preparation of the present invention does not irritate the patient's gastrointestinal track. Often solid particles or suspensions can irritate and/or actually ulcerate the tissue lining the gastrointestinal track. Surprising, the pharmaceutical preparation of the present invention was found not to induce any irritation and/or ulcers in tissue.

One or more of the additives of the present invention can be provided in tablet, pill, or capsule form all of which can include one or more binders, lubricants, and/or coatings. Specific examples of binders for use in the present invention include pharmaceutically accepted binders, such as cellulose and polyethylene glycol (PEG), gum tragacanth, acacia, cornstarch, or gelatin; potato starch, alginic acid and the like; a lubricant, such as magnesium stearate and the like. Preferably, the coating provides an enhanced route of administration and greater effectiveness. Preferred pharmaceutical compositions for the ease of preparation and administration includes solid compositions, particularly tablets that are hard-filled or liquid-filled capsules. It is preferred that the coating provides means for absorbing the serum toxins in the intestines, and particularly in the colon. This can include coatings that resist exposing the encapsulated additive from bodily fluids found in the stomach and from the harsh conditions such as the high acidity of the stomach. Preferably the additives are coated with a coating that does not dissolve in the stomach, but does dissolve or release the encapsulated sorbent/additive into the more basic environment found it the intestines and colon.

Such approaches may involve various types of controlled release systems, ranging from one, which may for example be based on a polymer, which simply provides a delayed release of the complex with time, through a system which is resistant to dissociation under acidic conditions, for example, by the use of buffering, to a system which and is biased towards release under conditions such as prevail in the small intestine, for example, a pH sensitive system which is stabilized towards a pH of 1 to 3 such as prevails in the stomach but not one of 7 to 9 such as prevails in the small intestine.

A particularly convenient approach to a controlled release composition involves encapsulating the zirconium-silicate sorbent in a material which is resistant to dissociation in the stomach but which is adapted towards dissociation in the small intestine. The preparation of solid composition adapted to resist dissociation under acidic conditions but adapted towards dissociation under non-acidic conditions is well known in the art and most often involves the use of enteric coating, whereby tablets, capsules, etc, or the individual particles or granules contained therein, are coated with a suitable material. Such procedures are described, for example, in the article entitled “Production of enteric coated capsules” by Jones in Manufacturing Chemist and Aerosol News, May 1970, and in such standard reference books as “Pharmaceutical Dosage Forms”, Volume III by Liebermann and Lackmann (published by Marcel Decker). One particular method of encapsulation involves the use of gelatine capsules coated with a cellulose acetate phthalateldiethylphthalate layer. This coating protects the gelatin capsule from the action of water under the acid conditions of the stomach where the coating is protonated and therefore stable. The coating is however destablished under the neutral/alkaline conditions of the intestine where it is not protonated, thereby allowing water to act on the gelatin. Other examples of methods of formulation which may be used include the use of polymeric hydrogel formulations which do not actually encapsulate the iron complex but which are resistant to dissociation under acidic conditions.

One or more of the components of the present invention can also be combined with a selected carrier, diluent, and/or adjuvant. The carriers are preferably pharmaceutically acceptable carriers that are commercially available. Examples of carriers include starch, lactose, di-calcium phosphate, microcrystalline cellulose, sucrose and kaolin. Liquid carries include sterile water, polyethylene glycols, non-ionic surfactants, and edible oils such as corn, peanut and sesame seed oils. Adjuvants customarily employed for the preparation of pharmaceutical compositions may advantageously be included, such as flavoring agents sucrose, lactose or saccharin peppermint, oil of wintergreen, or cherry flavoring, coloring agents, preserving agents and anti-oxidants, for example, Vitamin E, BHT and BHA. Dispersions can also be prepared in glycerol, liquid polyethylene glycols and mixtures of oils.

In preferred embodiments, the selected cation for zirconium-silicate sorbents are composed primarily of inorganic compounds rather than organic compounds. The organic compounds can provide less tendency to cause concretions causing bowel obstruction. For example, the zirconium-silicate sorbent can be used without an osmotic diarrheic without causing bowel obstruction or bowel irritation. Thus, the zirconium-silicate sorbents in accordance with the present invention may be administered without need to create diarrhea by administering non-absorbable saccharides. This can limit patient complaints about the bad taste, nausea and abdominal pain created by the non-absorbable saccharides. The sorbent can also be provided as a powder that can be suspended in water or in a milkshake or thick soup, or mixed into ice cream or a variety of other additives. Thus in accordance with the present invention, one or more of the additives can be combined with food to provide increased patient comfort and desirability to maintain the strict regime. Additionally, it may be possible to include one or more of the components to be baked into baked goods such as cookies, breads, and the like.

In order to treat patients, particularly patients that exhibit kidney or liver failure such as found, for example, in endstage renal disease, a considerable amount of the selective zirconium-silicate sorbent would need to be ingested or administered. The exact amount can be calculated by first deciding the percentage decrease in the serum level of toxin removal that would be beneficial for the patient. For example, assuming, particularly in endstage renal disease that the clearance of the toxin is near zero from the body, an intestinal absorbent must absorb a portion of the toxin generated every day that equals the desired decrease in serum concentration. From the expected intestinal concentration of this toxin and langmuir absorption curve, the amount required of sorbent to be ingested or administered can be calculated. For various applications the required amount of zirconium-silicate sorbent is considered to be between about 15 to 30 grams daily for hyperkalemia in kidney failure; between about 50 to 100 grams daily to decrease urea in kidney failure; and between about 30 to 60 grams daily for hyperammonemia for liver failure. Additionally, one or more of the components of the present invention can be administered to treat patients having elevated toxins from lithium overdose, excess plasma ammonium due to drugs such as DEPAKOTE as well as adverse interactions between prescribed medications.

For the purpose of promoting further understanding and appreciation of the present invention and its advantages, the following examples are provided. It will be understood, however, that these examples are illustrative and not limiting in any fashion.

### EXAMPLE 1

### Ammonium Binding Selectivity of Zirconium-Silicate Sorbent

In the preferred embodiment, the zirconium-silicate of the present invention exhibits an excellent selectivity for monovalent cations or with divalent cations. In specific examples, the zirconium-silicate combined to over ten times as much concentration ammonium rather than the zirconium phosphate. (The zirconium phosphate agent is described in U.S. Pat. No. 3,850,838 and in Nancollas, G. H. and Pekarek, V., “Sorption Properties of Zirconium Phosphates of Various Crystallinities”, J. Inorg. Nucl. Chem. vol. 27, 1409-1418, 1965, each is incorporated by reference in its entirety.) To perform these studies a solution of standard acetate dialysate was prepared. The solution contained physiologic concentrations of sodium, calcium, magnesium, potassium, chloride and was buffered (acetate) at neutral pH. To 35 ml of this dialysate solution was added 0.2 mM ammonium chloride and then 0.025 to 0.1 grams zirconium-silicate or zirconium phosphate. The resulting solution would represent a ratio of 50 to 210 grams of sorbent ingested by a 70 kg person. The resulting ammonium concentration was measured in the dialysate solution with suspended sorbents.

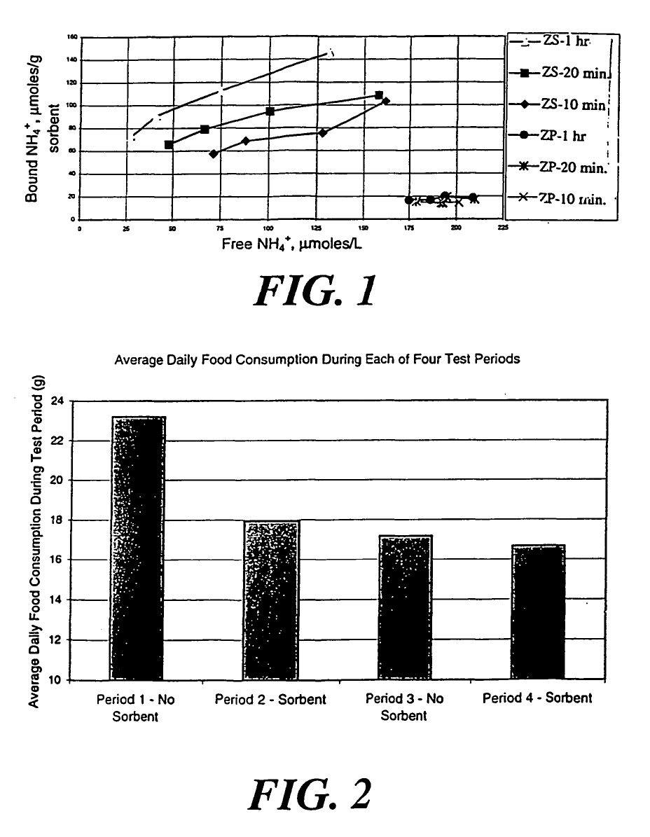

In FIG. 1, a graph illustrating the binding constant of the zirconium-silicate sorbent is illustrated. This graph indicates that zirconium-silicate has as much higher binding capacity for ammonium in this chemical environment than does commonly used zirconium phosphate. The ammonium binding of zirconium phosphate would be similar (on a per-gram basis) as commonly used oral agents such as PSS sold under the trade name KAYEXELATE® which is ascribed to adsorb potassium.

### EXAMPLE 2

### Efficacy of Removal of Ammonia and Potassium for the Rats by Monovalent-Selective Cation Exchanger as an Oral Sorbent

In order to determine the chemical effectiveness or absorbence in animals, an experiment was performed to balance the oral intake of various chemical compounds to output in urine or stool. It was decided to keep the intake the same and to measure the output of the chemicals in the urine during periods with sorbent congestion and periods without sorbent congestion. Laboratory experiments were conducted using four rats (approximately 300 grams each) which were used for a period of 20 days. The rats were fed a regular food diet (15 grams per day) allowing calculation of daily nitrogen intake. The animals were not divided for control and treatment group period. However, the experimental protocol was designed to be conducted over 20 days. The 20 day period was divided into four sections, each section lasting five days in length. During the first and third section, the rats were fed regular food. In those first and third sections of the experiments, testing rats were considered as a control group. In the second and fourth sections of the experiment, the rats were fed a diet that included the zirconium-silicate sorbent. A total 24 hour urine output and stool was collected for each rat. Urine levels of urea, nitrogen, potassium, calcium, as well as other electrolytes were measured on a daily basis.

Dried, powdered zirconium-silicate of 5-10 micron particle size was incorporated into the foodstuff (5001 Purina Rat Chow) at a concentration of 10% by weight based upon the total weight of the feed. The feed was then pelletized. The resulting pelletized feed had the same appearance and feel as normal pellets without the sorbent.

At the initiation of this study, 60.4 g of the sorbent supplement food was fed to a single rat to determine whether the supplemental food was edible by the rats. The rat was then placed in a metabolic cage for observation over two days. The rat food intake was stable and the rat showed no signs of visual physiological problems. Thereafter, four rats were used for the remaining of the study. All rats were male liter mates weighing approximately 300 g each, which were preacclimated to the metabolic cages prior to the start of the control.

All rats were observed during the testing periods and they exhibited a bright, alert and grooming procedure normal for their species. The sorbent diet was offered to each animal during the second and fourth time periods. Total urine and feces were collected daily from each animal and tested. During some of the collection, red spots were seen in the feces of some of the selected rats. The red spots were investigated, but tested negative for hemoglobin.

Experimental Section

Electrolytes (sodium, potassium, calcium, magnesium) and pH of urine were measured by means of Electrolyte 8 Analyzer from Nova Biomedical calibrated according to manufacturer instruction. Inorganic phosphorus was determined quantitatively by colorimetric method according to Sigma diagnostic kit (Procedure No. 670) and urea nitrogen according to Sigma procedure no. 640. Ammonia determination was based on enzymatic assay with glutamate dehydrogenase, oxoglutarate and NADPH. The decrease in absorbance at 340 nm due to oxidation of NADPH is proportional to the ammonia concentration. All above mentioned optical assays were performed by means Spectra Max Plus from Molecular Devices.

Zirconium-silicate sorbent binding during in vitro experiments revealed a high binding capacity of ammonia at various pH levels, in presence of calcium and magnesium. Absorption isotherms confirmed that the zirconium-silicate sorbent was able reduce ammonia level below levels associated with neurotoxicity. The sorbent retained its ability to bind ammonia after being integrated with food. Furthermore capacity of binding was not substantially affected by presence of calcium or magnesium in food or in dialysate. In conducted experiments, sorbent-foodstuff suspended in dialysate (physiologic salt solution including calcium and magnesium) containing approximately 140-150 μM/L of ammonia was able to bind 33-44 μmoles of ammonia per gram of sorbent. This was comparable to 47 μmole/g of sorbent when pure sorbent was tested in dialysate.

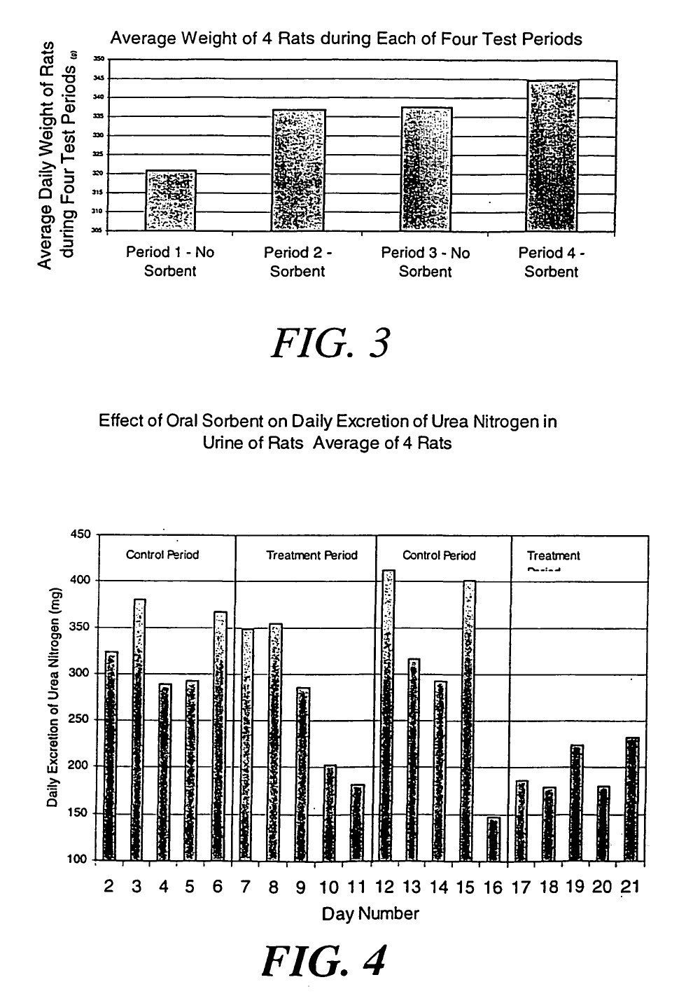

Four rats were used in this study of 20 days duration. In control periods one and three (5 days each) rats were fed regular rat food. In the intervals two and four, animal food with sorbent was supplied. Surprisingly the addition of 10% by weight of zirconium-silicate sorbent into the normal rat food did not decrease the amount of food intake per day for the rats (FIG. 2). Rats maintained weight essentially at the same level during the trial (FIG. 3). The zirconium-silicate sorbent loaded food was obviously palatable to the rats. The only visible change in stool character during the study was the occasional appearance of “red spots” within the stool. These spots were not due to blood since Hemoccult tests of the stool were negative. It is possible that iron binding of the zirconium-silicate sorbent caused these occasional red flecks in the stool. Gross post-mortem analysis of the rats was entirely normal, as was the histologic appearance of the mucose of the small bowel and large bowel.

This study revealed that the zirconium-silicate sorbent in food was capable of removing ammonia as was checked by level of urea nitrogen in urine of experimental animals. Within the first oral sorbent phase urea nitrogen in urine dropped by 10% compared to prior control 5 days. This urine urea level plunged even more drastically by approximately 45% during second sorbent phase (FIG. 4). The function of the sorbent in the gut in removing urea can be compared to the kidney. Both the gut and kidney receive the same blood within the animal, and therefore are in essence in competition for the urea and the nitrogen that results from urea breakdown (fraction of solute removed by sorbent clearance of sorbent/clearance of kidneys). In the first phase of the study, the sorbent within the gut removed per day 10% as much urea as the kidney, and therefore the clearance rate (efficiency) of urea/ammonium removal by the sorbent in the gut represented 11% of the clearance by the normal kidneys. In the third phase, the sorbent clearance rate was 81% of that of the clearance by the normal kidneys.

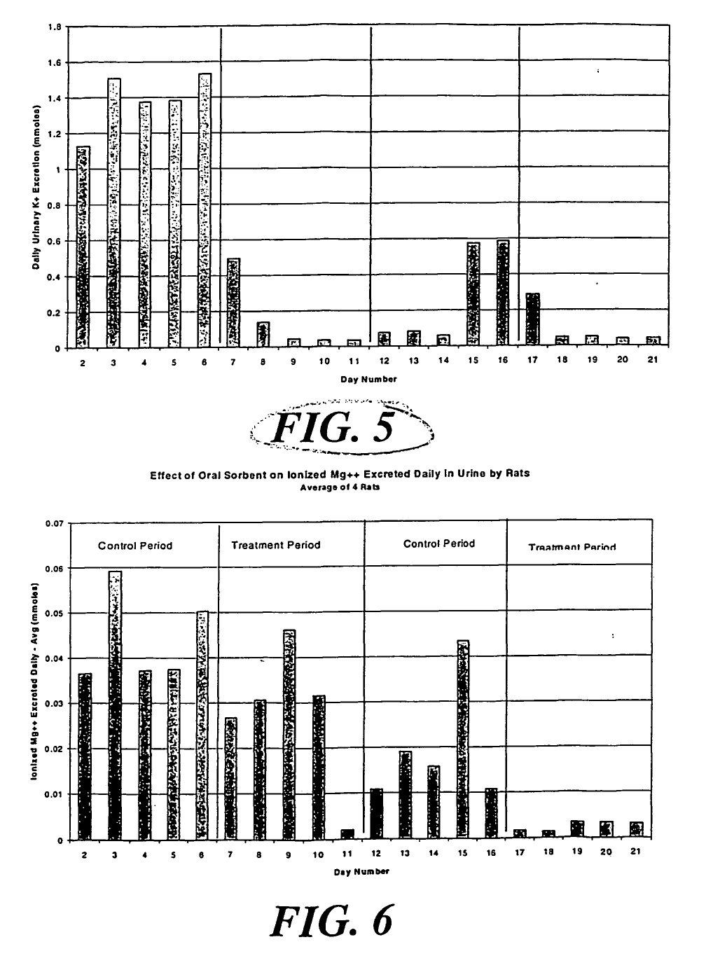

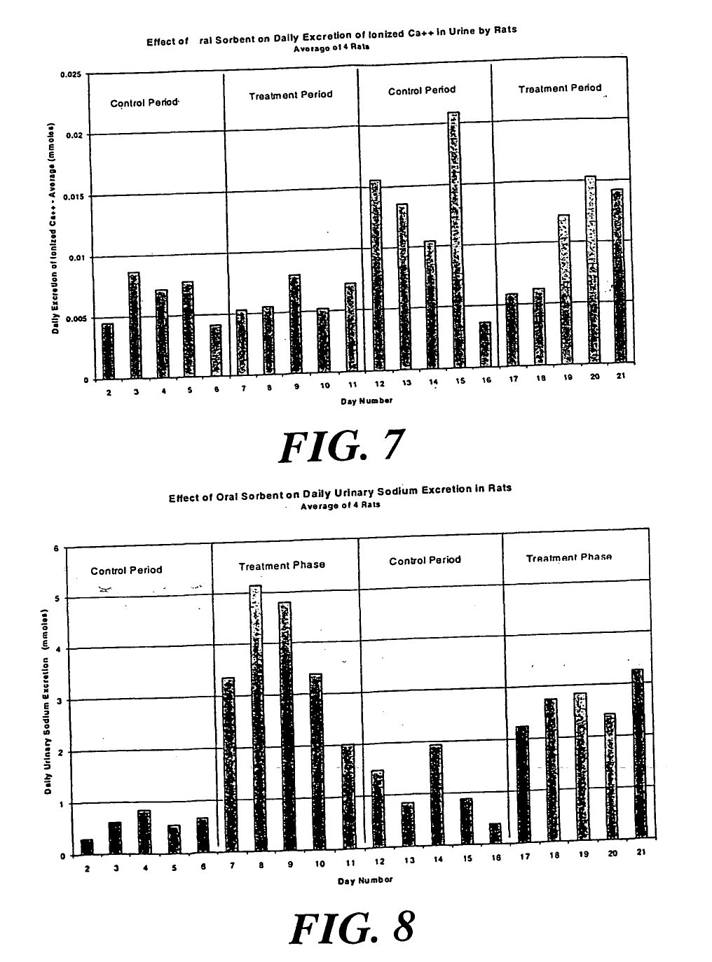

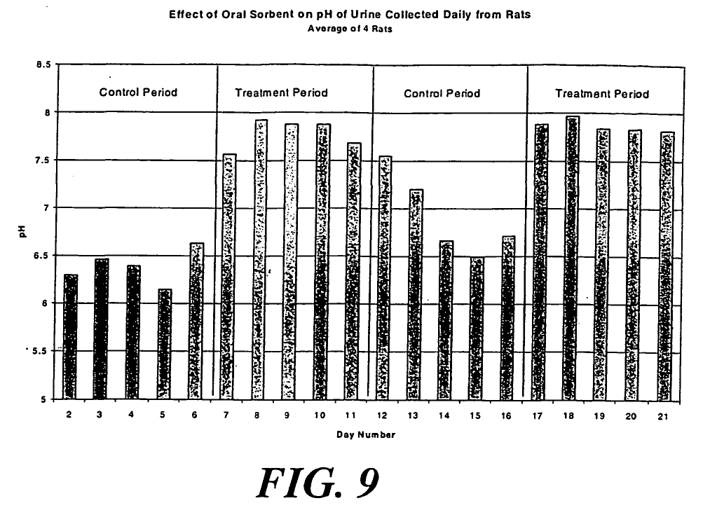

The level of some urinary electrolytes was also reduced during oral sorbent therapy, mainly potassium and magnesium. Removal of potassium was very fast at the start of each sorbent phase and in fact approached zero in each phase, meaning that all of the potassium in the diet was prevented from entering the rat (FIG. 5). Removal of magnesium was less by percentage than potassium and occurred faster in second phase of oral sorbent treatment (FIG. 6). A minimal tendency of removal of calcium was found (FIG. 7). Because of the nature of sorbent loading it was found an increased level of sodium in the urine (FIG. 8) and pH in the urine (FIG. 9). The zirconium-silicate sorbent used in the study was 100% sodium-loaded. Therefore the release of sodium equal to the binding of all other cations was not unexpected. The release sodium ion from sorbent probably was responsible for increase volume of urine (through also an increased drinking of water due to the normal thirst mechanism). The increase in urine pH occurred because hydrogen was bound by the zirconium-silicate sorbent, again in exchange for sodium (FIG. 9). Unexpectedly, the amount of sodium contributed to the animals and excreted through the urine was much less, on a molar basis, than the amount of cations removed from the animal by the zirconium-silicate. The increase in sodium excretion during feeding of the zirconium-silicate was about 3 millimoles per day. However, the removal of calcium, magnesium, potassium, urea nitrogen (1 mM per 14 mg) and hydrogen (1 mM per pH unit urine increase) totaled 12.4 mM per day. Thus, though use of purely sodium-loaded zirconium-silicate as an oral sorbent would result in sodium absorption by patients with kidney failure or liver failure, the sodium load would not be as great as might be expected. Partially loading the zirconium-silicate with hydrogen or calcium would further diminish sodium load in these patients.

There are various applications that are possible for zirconium-silicate sorbent (or similar monovalent-selective cation exchangers) as an oral sorbent. Depending upon the disease state being treated, the zirconium-silicate sorbent could be loaded with differing cations. The zirconium-silicate sorbent can be loaded with any number of monovalent cations and still remove ammonium. For treatment of hyperkalemia in patients with some kidney function, sodium loading would be advantageous since the absorbed sodium would increase urine flow and by this increase kidney excretion of potassium. For treatment of chronic kidney insufficiency to remove urea and potassium, sodium release would only add to the sodium and fluid overload of the patients. The zirconium-silicate sorbent loading with calcium and hydrogen would be advantageous. The calcium released would bind phosphate within the gut. The hydrogen released would balance an increase in pH by the generation of carbonate from urease within the gut bacteria Urease is an enzyme that produces ammonium and carbonate from urea Since urease is product-limited, removing ammonium and decreasing local pH increases the rate of the urease reaction. For treatment of conditions of hyperammonemia (such as symptomatic in liver dysfunction or failure) sodium release would also be problematic, since these patients also have sodium excess. Loading with calcium and hydrogen would be appropriate.

The present invention contemplates modifications as would occur to those skilled in the art. It is also contemplated that embodiments of the present invention can be altered, rearranged, substituted, deleted, duplicated, combined or added to other processes or treatment methods as would occur to those skilled in the art without departing from the spirit of the present invention. In addition, various procedures, techniques and treatment methods that are within those processes may be altered, rearranged, substituted, deleted, duplicated or combined. All publications, patents and patent applications cited in the specification are herein incorporated by reference as if each individual publication, patent or patent application were specifically and individually indicated to be incorporated by reference and set forth in its entirety herein. Further, any theory of operation, proof or finding stated herein is meant to further enhance understanding of the present invention and is not intended to make the scope of the present invention dependent upon such theory, proof or finding.

While the invention has been illustrated and described in detail in the drawings and foregoing description, the same is considered to be illustrative and not restrictive in character, it is understood that only the preferred embodiments have been shown and described and that all changes and modification that come within the spirit of the invention are desired to be protected.

What is claimed is:

1. A method of treating animals said method comprising:
selecting an animal capable of deriving a benefit from lower levels of one or more serum toxins;
administering to said animal a pharmaceutical preparation comprising a zirconium-silicate sorbent having exchangeable cations including hydronium cations and one or more cations selected from the group consisting of: calcium, sodium, potassium magnesium and mixtures thereof adsorbed thereon and provided to selectively adsorb said one or more toxins.

2. The method of claim 1 wherein the zirconium-silicate sorbent comprises hydronium cations and calcium cations.

3. The method of claim 1 wherein the pharmaceutical preparation comprising one or more of charcoal, zinc oxide and an intestinal tissue permeability enhancing agent.

4. The method of claim 3 wherein the pharmaceutical preparation comprises between about 0.1 g and about 3 g of charcoal per kilogram of body weight.

5. The method of claim 3 wherein the pharmaceutical preparation comprises between about 10 mg/L solution and about 1000 mg/L of a non absorbable alcohol per kilogram of body weight.

6. The method of claim 4 wherein the pharmaceutical preparation comprises between about 10 mg/L solution and about 1000 mg/L of a non absorbable alcohol per kilogram of body weight.

7. The method of claim 1 wherein said administering comprising administrating one or more of: charcoal, zinc oxide and an intestinal tissue permeability enhancing agent.

8. The method of claim 7 wherein said administering comprising administering between about 0.1 g and about 3 g of charcoal per kilogram of body weight.

9. The method of claim 7 wherein said administering comprising administering between about 0.05 g and about 5 g of zinc oxide per kilogram of body weight.

10. The method of claim 8 wherein said administering comprising administering between about 0.05 g and about 5 g of zinc oxide per kilogram of body weight.

11. The method of claim 1 wherein the animal exhibits, at least one of hyperkalemia, hypercalcemia, hypermagnesemia, hypernatremia, hyperammonemia, hyperphosphatemia, or uremia

12. The method of claim 1 wherein said administering comprises orally administering said sorbent.

13. The method of claim 1 wherein the pharmaceutical preparation is provided as a pill, a pellet, a gel or a liquid suspension.

14. The method of claim 1 wherein said administering comprises admixing said sorbent with food.

15. The method of claim 1 comprising administering the pharmaceutical preparation through a ostomy inlet into a patient's gasterointestinal system.

16. The method of claim 1 wherein said administering comprising administering a therapeutically effective amount of the sorbent selected to be between about 0.05 g and about 1.5 g per kilogram of body weight daily.

17. The method of claim 16 wherein the therapeutically effective amount is selected to be between about 0.15 g and about 0.5 g per kilogram of body weight daily.

18. A method of treating renal or kidney dysfunction, said method comprising:
administering to a patient either in combination or separately a pharmaceutical preparation comprising a zirconium-silicate sorbent having exchangeable cations including cations selected from the group consisting of: hydronium, calcium, sodium, potassium, magnesium and mixtures thereof; and
an intestinal permeability enhancing agent.

19. The method of claim 18 wherein said pharmaceutical preparation is administered orally or rectally.

20. The method of claim 18 wherein the intestinal permeability enhancing agent is administered into the gastrointestinal tract.

21. The method of claim 20 wherein the intestinal permeability enhancing agent is selected from the group consisting of: ethanol, polyethylene glycol, glycerin, propylene glycol, acetone, and polyvinyl alcohol and mixtures thereof.

22. The method of claim 18 wherein the pharmaceutical preparation comprises charcoal.

23. The method of claim 18 wherein the pharmaceutical preparation comprises zinc oxide.

24. The method of claim 22 wherein the pharmaceutical preparation comprises zinc oxide.

25. The method of claim 18 wherein said administrating comprises administering a composition comprising charcoal.

26. The method of claim 18 wherein said administrating comprises administering a composition comprising zinc oxide.

27. The method of claim 25 wherein said administrating comprises administering a composition comprising zinc oxide.

28. The method of claim 18 wherein administering comprises administering through a ostomy inlet.

29. A pharmaceutical composition comprising:
a cation exchanger comprising a zirconium-silicate sorbent having exchangeable cations including cations selected from the group consisting of: hydronium, calcium, sodium, potassium, magnesium and mixtures thereof; in a therapeutically effective amount to reduce a serum concentration of one or more serum toxins, and
at least one therapeutic additive selected from activated charcoal and an intestinal tissue permeability-enhancing agent.

30. The pharmaceutical composition of claim 29 wherein the therapeutically effective amount is between about 0.15 g and about 1.5 g per kilogram of body weight.

31. The pharmaceutical composition of claim 29 wherein the monovalent cation exchanger comprises a mixture of calcium and hydronium cations.

32. The pharmaceutical composition of claim 29 comprising between 0.1 g and about 3 g of charcoal per kilogram of body weight.

33. The pharmaceutical composition of claim 29 comprising between 10 mg/L solution and about 1000 mg/L of a non absorbable alcohol per kilogram of body weight.

34. The pharmaceutical composition of claim 32 comprising between 10 mg/solution and about 1000 mg/L of a non absorbable alcohol per kilogram of body weight.

35. The pharmaceutical composition of claim 29 comprising zinc oxide.

36. The pharmaceutical composition of claim 32 comprising zinc oxide.
